SEQUENCE LISTING (1 sequence)

SEQ ID NO: 1 - protein, 572 aa, Bacteriophage phi-29
MPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAERLLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRVKYLRQKTYIQDIYMKEVDGKLVEGSPDDYTDIKFSVKCAGMTDKIKKEVTFENFKVGFSRKMKPKPVQVPGGVVLVDDTFTIK

(19) United States
(12) Patent Application Publication
(10) Pub. No.: US 2012/0178092 A1
(43) Pub. Date: Jul. 12, 2012

(54) Method for the Replication, Amplification or Sequencing of a DNA Template

(75) Inventors: Margarita Salas Falgueras, Madrid (ES); Miguel De Vega Jose, Madrid (ES); Jose M. Lazaro Bolos, Madrid (ES); Luis Blanco Davila, Madrid (ES); Mario Mencia Caballero, (US)

(73) Assignee: CONSEJO SUPERIOR DE INVESTIGACIONES CIENTIFICAS, Madrid (ES)

(21) Appl. No.: 13/381,845

(22) PCT Filed: Jul. 2, 2010
(86) PCT No.: PCT/ES2010/070456

§ 371 Date: Mar. 21, 2012

(30) Foreign Application Priority Data
Jul. 2, 2009 (ES) P 200930412

(51) Int. Cl. C12Q 1/68; C12N 9/12; C12P 19/34

(52) U.S. Cl. 435/6.12; 435/91.2; 435/6.1; 435/91.5; 435/194

(57) ABSTRACT

Methods for replicating, amplifying or sequencing a deoxyribonucleic acid with a φ29 type DNA polymerase are disclosed, along with kits for carrying out the methods.

### CROSS REFERENCE TO RELATED APPLICATIONS

This application is a national stage of

International Application No. PCT/ES2010/070456, filed Jul. 2, 2010, which claims priority under 35 U.S.C. §119 to Spanish Patent Application No. P 200930412, filed Jul. 2, 2009, the entire disclosure of which is herein expressly incorporated by reference.

### SEQUENCE LISTING

The instant application contains a Sequence Listing which has been submitted via EFS-Web as an ASCII text file and is hereby incorporated by reference in its entirety. Said ASCII file, created on Dec. 30, 2011, is named SEQ22328_{—}0012US.txt and is 5,201 bytes in size.

### FIELD OF THE INVENTION

The present invention is encompassed within the biotechnology field. Specifically, it relates to a method for replicating, amplifying or sequencing a deoxyribonucleic acid with a φ29 type DNA polymerase and to a kit for carrying out said method.

### BACKGROUND AND SUMMARY

The only enzyme required by the bacteriophage φ29 to replicate its genome is its DNA polymerase, a 66 KDa monomeric protein capable of catalyzing both the initiation of the replication and the elongation of the synthesized strand. For the initiation, this polymerase is bound to a protein known as “terminal” (TP), recognizes the end of the φ29 DNA and catalyzes the formation of a TP-dAMP covalent complex. After the polymerization of 10 nucleotides, the DNA polymerase/TP heterodimer disassociates and the elongation of the strand coming from DNA is carried out.

Replicative DNA polymerases require the interaction with accessory proteins which stabilize the binding between the enzyme and the DNA (Kuriyan and O'Donnell. J Mol Biol. 1993; 234: 915-925). On the other hand, said DNA polymerases need to couple the polymerization upon the detachment of the DNA strand which is not being copied for which they require the functional association thereof to helicase type proteins. In this sense, the DNA polymerase of the bacteriophage φ29 has various intrinsic functional characteristics making it unique:
a) High processivity (defined as the number of nucleotides incorporated by binding event).
b) High strand detachment capacity which allows replicating the genome of said bacteriophage in the absence of helicase type accessory proteins. These two characteristics, processivity and strand detachment allow the φ29 DNA polymerase to be capable of synthesizing DNA strands of more than 70 kb in length (Blanco et al. J Biol Chem. 1989; 264: 8935-8940).
c) High accuracy in the insertion of nucleotides in the new strand (Esteban et al. J Biol Chem. 1993; 268: 2719-2726).

All these characteristics have led to the development of a great variety of isothermal process (at constant temperature) protocols for amplifying double stranded DNA (dsDNA) based on the use of this polymerase. In a simple configuration, the capacity of the φ29 DNA polymerase to use circular single stranded DNA (ssDNA) allows amplifying DNA by the rolling circle method (or RCA—rolling-circle amplification), producing ssDNA molecules of great length and containing more than 10 copies of the circular template (Blanco et al. J Biol Chem. 1989; 264: 8935-8940; U.S. Pat. No. 5,001,050, U.S. Pat. No. 5,198,543 and U.S. Pat. No. 5,576,204). In the process for amplifying dsDNA developed by Amersham Biosciences/Molecular Staging (Dean et al. Genome Res. 2001; 11: 1095-1099; Dean et al. Proc Natl Acad Sci USA. 2002; 99: 5261-5266), the combination of the use of the φ29 DNA polymerase with the use of hexamers (hexa-nucleotides) random sequence primers allows obtaining amplification factors of 10⁴-10⁶ starting from picograms of circular plasmid DNA [Templiphi™ of GE Healthcare] or from 10 nanograms of Genomic DNA [Genomiphi™ of GE Healthcare and Repli-G® of Qiagen]. The products produced are of high quality and can be digested or sequenced directly without the need of prior purification, it has been demonstrated that the φ29 DNA polymerase is the most robust enzyme for this purpose. The common buffer for carrying out the amplification reactions with the φ29 DNA polymerase contains tris-HCl (pH 7.5) plus different concentrations (in the millimolar order) of NaCl or KCl and MgCl₂ (US20030207267). However, in spite of the satisfactoriness of these protocols in very diverse situations, the development of other protocols which allow starting from lesser DNA amounts is a growing need.

The present invention relates to a method for replicating, amplifying or sequencing a deoxyribonucleic acid with a φ29 type DNA polymerase and to a kit for carrying out said method.

The phage φ29 DNA polymerase has several characteristics of great interest for amplifying DNA such as: a high processivity without the need of the participation of any accessory protein and a high strand detachment capacity allowing it to replicate the genome of said bacteriophage in a single binding event to the DNA, as well as a high accuracy in the insertion of nucleotides in the new strand. These characteristics have lead to the development of a great variety of protocols for the isothermal amplification of DNA based on the use of this polymerase which allow obtaining products of high quality that can be digested or sequenced directly without the need of prior purification. However, there is a need for protocols which allow the amplification of DNA from lesser amounts thereof. The present invention responds to this need by means of developing a method for amplifying DNA which significantly improves the specificity and the yield of the reaction.

In the examples of this patent, it is shown that the simultaneous addition of polyoxyethylenated sorbitan monolaurate (Tween®20) and an ammonium salt to the buffer commonly used for the amplification with the φ29 DNA polymerase, on the one hand, prevents the non-specific DNA amplification and, on the other hand allows the detectable and specific amplification from limited amounts of φ0.1 femtograms (fg) of plasmid DNA and 10 fg of genomic DNA as template.

A first aspect of the present invention relates to a method for replicating, amplifying or sequencing a template DNA which comprises contacting said DNA with a reaction mixture comprising at least:
a) a φ29 type DNA polymerase,
b) polyoxyethylenated sorbitan monolaurate,
c) an ammonium salt,
d) a buffer,
e) magnesium chloride,
f) a primer, and
g) nucleoside triphosphates.

A preferred embodiment of this aspect of the invention relates to a method for replicating, amplifying or sequencing a template DNA which comprises contacting said DNA with a reaction mixture comprising the aforementioned elements (a)-(g) and further comprising a potassium salt. Preferably, said potassium salt is potassium chloride or potassium acetate.

### BRIEF DESCRIPTION OF THE DRAWINGS

Features and advantages of the present invention will become apparent to those skilled in the art from the following description with reference to the drawings, in which:

FIG. 1 shows the effect of Tween® 20 and (NH4)₂SO₄ in the amplification capacity of the φ29 DNA polymerase. The assay was carried out as described in the main text in the presence of the indicated amounts of plasmid DNA (4.2 kpb). After incubating at 30° C. for 5 h, the reactions were analyzed as described in the main text. On the left, the linear DNA fragments obtained after digesting the φ29 DNA with HindIII used as DNA length markers.

FIG. 2 shows the amplification of different amounts of plasmid DNA (in the order of femtograms) by the φ29 DNA polymerase in the presence of Tween® 20 and (NH4)₂SO₄. The assay was carried out as described in the main text in the presence of φ0.025% Tween®20 and of 45 mM (NH4)₂SO₄. The DNA length markers are the same as those used in FIG. 1.

FIG. 3 shows the effect of the NH₄⁺ ion in the amplification capacity of the φ29 DNA polymerase. The assay was carried out as described in the main text in the presence of φ0.025% Tween® 20 and the indicated ammonium salt as well as the indicated amounts of plasmid DNA (4.2 kpb). After incubating at 30° C. for 6 h, the reactions were analyzed as described in the main text. The DNA length markers are the same as those used in FIG. 1.

FIG. 4 shows the amplification of different amounts of Bacillus subtilis genomic DNA by the φ29 DNA polymerase in the presence of Tween® 20 and (NH4)₂SO₄. The assay was carried out as described in the main text in the presence of φ0.025% Tween® 20 and 45 mM (NH4)₂SO₄. The DNA length markers are the same as those used in FIG. 1.

FIG. 5 shows the significant improvement depicted by the addition of φ0.025% Tween® 20 and 45 mM (NH₄)₂SO₄ to the current reaction buffer of a commercial kit for the amplification of DNA based on the φ29 DNA polymerase (Illustra kit of General Electrics HealthCare). The assay was carried out as described in the main text. The DNA length markers are the same as those used in FIG. 1.

### DETAILED DESCRIPTION

For simplicity and illustrative purposes, the principles of the present invention are described by referring to various examples. One of ordinary skill in the art will readily recognize that the same principles are equally applicable to, and can be implemented in other forms, and that any such variation would be within those modifications that do not part from the true spirit and scope of the present invention. The invention is not limited in its application to the details of any particular formulation shown, since the invention is capable of other embodiments. The following examples are provided for illustrative purposes and do not and should not be understood to limit the claims appended hereto. The terminology used herein is for the purpose of description and not of limitation.

As used in the present description, the term “DNA polymerase” relates to an enzyme capable of catalyzing the polymerization of deoxynucleoside triphosphates. Generally, the enzyme initiates the synthesis in the 3′ end of a primer hybridized with a template DNA sequence and proceeds towards the 5′ end of the template DNA strand.

As used in the present invention, the term “φ29 type DNA polymerase” relates to any DNA polymerase containing TPR1 and TPR2 subdomains in its polymerization domain providing the polymerase with the capacity of coupling the processive polymerization to the strand detachment. Examples of φ29 type DNA polymerases that can be used in the present invention are selected from the list comprising the DNA polymerases isolated from the following phages: φ29, Cp-1, PRD-1, φ15, φ21, PZE, PZA, Nf, M2Y, B103, GA-1, SF5, Cp-5, Cp-7, PR4, PR5, PR722, L17 or Acidianus Bottle-shaped virus (ABV).

In a preferred embodiment of the method for replicating, amplifying or sequencing of the invention, the φ29 type DNA polymerase is selected from the DNA polymerases isolated from the following phages: φ29, Cp-1, PRD-1, φ15, φ21, PZE, PZA, Nf, M2Y, B103, GA-1, SF5, Cp-5, Cp-7, PR4, PR5, PR722, L17 or Acidianus Bottle-shaped virus (ABV).

In a preferred embodiment of the method for replicating, amplifying or sequencing of the invention, the φ29 type DNA polymerase has an amino acid sequence having an identity of at least 80% with SEQ ID NO: 1. In a more preferred embodiment, the φ29 type DNA polymerase has an amino acid sequence having an identity of at least 90% with SEQ ID NO: 1. In a still more preferred embodiment, the φ29 type DNA polymerase has the amino acid sequence SEQ ID NO: 1.

The exonuclease domain of the φ29 type DNA polymerases is known and can be modified to reduce the exonuclease activity retaining a high processivity and strand detachment capacity. These modified DNA polymerases are especially useful for sequencing large molecules.

In a preferred embodiment of the method for replicating, amplifying or sequencing of the invention, the φ29 type DNA polymerase has a modification in the exonuclease domain, wherein said modified DNA polymerase has less than 10% of exonuclease activity than the corresponding naturally occurring DNA polymerase or “wild type”. In a more preferred embodiment, the modified φ29 type DNA polymerase has less than 1% of exonuclease activity than the corresponding naturally occurring DNA polymerase. In a still more preferred embodiment, the modified φ29 type DNA polymerase lacks detectable exonuclease activity with respect to the corresponding naturally occurring DNA polymerase.

In a preferred embodiment of the method for replicating, amplifying or sequencing of the invention, the φ29 type DNA polymerase (natural or modified in the exonuclease domain) is at a concentration between 5 nM and 75 nM. In a more preferred embodiment, the φ29 type DNA polymerase (natural or modified in the exonuclease domain) is at a concentration between 25 nM and 60 nM. In a still more preferred embodiment, the φ29 type DNA polymerase (natural or modified in the exonuclease domain) is at a concentration of approximately 50 nM.

In a preferred embodiment of the method for replicating, amplifying or sequencing of the invention, the polyoxyethylenated sorbitan monolaurate (Tween® 20) is at a concentration between φ0.003% and φ0.1% of the total volume of the reaction. In a more preferred embodiment, the polyoxyethylenated sorbitan monolaurate is in a proportion between φ0.006% and φ0.05% of the total volume of the reaction. In a still more preferred embodiment, the polyoxyethylenated sorbitan monolaurate is in a proportion between φ0.01% and φ0.03% of the total volume of the reaction. In a still more preferred embodiment, the polyoxyethylenated sorbitan monolaurate is in a proportion of approximately φ0.025% of the total volume of the reaction. “Total volume of the reaction” is understood as the resulting volume after the addition of the template DNA to the reaction mixture.

In a preferred embodiment of the method for replicating, amplifying or sequencing of the invention, the ammonium salt is selected from the list comprising: ammonium sulfate, ammonium chloride or ammonium acetate.

In a preferred embodiment of the method for replicating, amplifying or sequencing of the invention, the ammonium salt is ammonium sulfate. In a more preferred embodiment, the ammonium sulfate is at a concentration between 30 mM and 60 mM. In a still more preferred embodiment, the ammonium sulfate is at a concentration between 40 mM and 50 mM. In a still more preferred embodiment, the ammonium sulfate is at a concentration of approximately 45 mM.

In a preferred embodiment of the method for replicating, amplifying or sequencing of the invention, the ammonium salt is ammonium chloride. In a more preferred embodiment, the ammonium chloride is at a concentration between 60 mM and 120 mM. In a still more preferred embodiment, the ammonium chloride is at a concentration between 80 mM and 100 mM. In a still more preferred embodiment, the ammonium chloride is at a concentration of approximately 90 mM.

In a preferred embodiment of the method for replicating, amplifying or sequencing of the invention, the ammonium salt is ammonium acetate. In a more preferred embodiment, the ammonium acetate is at a concentration between 60 mM and 120 mM. In a still more preferred embodiment, the ammonium acetate is at a concentration between 80 mM and 100 mM. In a still more preferred embodiment, the ammonium acetate is at a concentration of approximately 90 mM.

In a preferred embodiment of the method for replicating, amplifying or sequencing of the invention, the buffer is at a pH between 7.0 and 8.5. In a more preferred embodiment, the buffer is at a pH between 7.2 and 8. In a still more preferred embodiment, the buffer is at a pH of approximately 7.5.

In a preferred embodiment of the method for replicating, amplifying or sequencing of the invention, the buffer is tris-hydrochloric, tris-acetic or HEPES. In a more preferred embodiment of the method for replicating, amplifying or sequencing of the invention, the buffer tris-hydrochloric, tris-acetic or HEPES is at a pH between 7.0 and 8.5. In a still more preferred embodiment, the buffer tris-hydrochloric, tris-acetic or HEPES is at a pH between 7.2 and 8. In a still more preferred embodiment, the buffer tris-hydrochloric, tris-acetic or HEPES is at a pH of approximately 7.5.

In a preferred embodiment of this aspecto of the method for replicating, amplifying or sequencing of the invention, the buffer tris-hydrochloric, tris-acetic or HEPES is at a concentration between 25 mM and 50 mM. In a more preferred embodiment, the buffer tris-hydrochloric, tris-acetic or HEPES is at a concentration between 30 mM and 45 mM. In a still more preferred embodiment, the buffer tris-hydrochloric, tris-acetic or HEPES is at a concentration of approximately 40 mM.

In a preferred embodiment of the method for replicating, amplifying or sequencing of the invention, the potassium chloride or the potassium acetate is at a concentration between 30 mM and 70 mM. In a more preferred embodiment, the potassium chloride or the potassium acetate is at a concentration between 40 mM and 60 mM. In a still more preferred embodiment, the potassium chloride or the potassium acetate is at a concentration of approximately 50 mM.

In a preferred embodiment of the method for replicating, amplifying or sequencing of the invention, the magnesium chloride is at a concentration between 2 mM and 20 mM. In a more preferred embodiment, the magnesium chloride is at a concentration between 5 mM and 15 mM. In a still more preferred embodiment, the magnesium chloride is approximately 10 mM.

In a preferred embodiment of the method for replicating, amplifying or sequencing of the invention, the polyoxyethylenated sorbitan monolaurate is in a proportion between φ0.01% and φ0.03% of the total volume, the ammonium sulfate is at a concentration between 40 mM and 50 mM, the buffer tris-hydrochloric, tris-acetic or HEPES is at a concentration between 30 mM and 45 mM and at a pH between 7.2 and 8.0, the magnesium chloride is at a concentration between 5 mM and 15 mM and the potassium chloride or the potassium acetate is at a concentration between 40 mM and 60 mM.

In a preferred embodiment of the method for replicating, amplifying or sequencing of the invention, the polyoxyethylenated sorbitan monolaurate is in a concentration of φ0.025% of the total volume, the ammonium sulfate is at a concentration of 45 mM, the buffer tris-hydrochloric, tris-acetic or HEPES is at a concentration of 40 mM and at a pH of 7.5, the magnesium chloride is at a concentration of 10 mM and the potassium chloride or the potassium acetate is at a concentration of 50 mM.

In a preferred embodiment of the method for replicating, amplifying or sequencing of the invention, the polyoxyethylenated sorbitan monolaurate is in a proportion between φ0.01% and φ0.03% of the total volume, the ammonium chloride is at a concentration between 80 mM and 100 mM, the buffer tris-hydrochloric, tris-acetic or HEPES is at a concentration between 30 mM and 45 mM and at a pH between 7.2 and 8.0, the magnesium chloride is at a concentration between 5 mM and 15 mM and the potassium chloride or the potassium acetate is at a concentration between 40 mM and 60 mM.

In a more preferred embodiment of the method for replicating, amplifying or sequencing of the invention, the polyoxyethylenated sorbitan monolaurate is in a concentration of φ0.025% of the total volume, the ammonium chloride is at a concentration of 90 mM, the buffer tris-hydrochloric, tris-acetic or HEPES is at a concentration of 40 mM and at a pH of 7.5, the magnesium chloride is at a concentration of 10 mM and the potassium chloride or the potassium acetate is at a concentration of 50 mM.

In a preferred embodiment of the method for replicating, amplifying or sequencing of the invention, the polyoxyethylenated sorbitan monolaurate is in a proportion between φ0.01% and φ0.03% of the total volume, the ammonium acetate is at a concentration of between 80 mM and 100 mM, the buffer tris-hydrochloric, tris-acetic or HEPES is at a concentration between 30 mM and 45 mM and at a pH between 7.2 and 8.0, the magnesium chloride is at a concentration between 5 mM and 15 mM and the potassium chloride or the potassium acetate is at a concentration between 40 mM and 60 mM.

In a more preferred embodiment of the method for replicating, amplifying or sequencing of the invention, the polyoxyethylenated sorbitan monolaurate is in a concentration of φ0.025% of the total volume, the ammonium acetate is at a concentration of 90 mM, the buffer tris-hydrochloric, tris-acetic or HEPES is at a concentration of 40 mM and at a pH of 7.5, the magnesium chloride is at a concentration of 10 mM and the potassium chloride or the potassium acetate is at a concentration of 50 mM.

As used in the present description, the term “replication” relates to the synthesis of a complementary DNA from a template DNA.

As used in the present description, the term “amplification” relates to the increase of the number of copies of a template DNA.

As used in the present description, the term “sequencing” relates to the determination of the order of the nucleotides of a template DNA.

“Contacting” is understood as the fact that the template DNA and the reaction mixture are incubated in primer extension conditions.

As used herein, the term “primer” relates to a oligonucleotide capable of acting as the starting point of the DNA synthesis when it is in primer extension conditions. Preferably, the primer is a deoxyribose oligonucleotide.

The primers can be prepared by means of any suitable method, including for example, but not limited to, the direct chemical synthesis. The primers can be designed to hybridize with specific deoxynucleotide sequences in the template DNA (specific primers) or can be randomly synthetized (arbitrary primers).

As used in the present description, the term “specific primer” relates to a primer the sequence of which is complementary to a specific deoxynucleotide sequence in the template DNA to be amplified.

“Complementary” is understood as the fact that the primer can be hybridized with a region of the template DNA such that it can act as the starting point of the DNA synthesis when it is in primer extension conditions. Preferably, that region has a 100% complementarity with a region of the template DNA. In other words, each nucleotide in the region of complementarity with the primer can form hydrogen bonds with a nucleotide present in the single stranded template. However, those with a normal experience in the field will acknowledge that primers having a region with less than 100% complementarity with respect to the template DNA will function to carry out the method for replicating, amplifying or sequencing of the present invention.

The term “arbitrary primer” relates to a primer the sequence of which is randomly synthesized and which is used to initiate the DNA synthesis in random positions of the template DNA. Generally, in the method for replicating, amplifying or sequencing of the present invention a population of arbitrary primers is used. The term “arbitrary primers” relates to a set of primers with a random sequence and which are used to initiate the DNA synthesis in random positions of the template DNA.

In a preferred embodiment of the method for replicating, amplifying or sequencing of the invention, the primer is specific.

In another preferred embodiment of the method for replicating, amplifying or sequencing of the invention, the primer is arbitrary. Preferably, the arbitrary primer is protected against the action of 3′-5′ exonucleases. And more preferably, the arbitrary primer is an oligonucleotide of 6 nucleotides, “hexanucleotide” or “hexamer” protected against the action of 3′-5′ exonucleases.

As used in the present description, the expression “protected against the action of exonucleases” relates to a modified primer such that it is resistant to the nucleolytic degradation by any 3′-5′ exonuclease activity present in the DNA polymerase.

In the method for replicating, amplifying or sequencing of the invention, more than one primer can be used, being able to use specific and/or arbitrary primers.

In a preferred embodiment of the method for replicating, amplifying or sequencing of the invention, the primer is at a concentration between 2 μM and 100 μM. In a more preferred embodiment, the primer is at a concentration between 20 μM and 80 μM. In a still more preferred embodiment, the primer is at a concentration between 40 μm and 60 μM. In a still more preferred embodiment, the primer is at a concentration of approximately 50 μM.

As used in the present description the term “nucleoside triphosphates” relates to organic molecules formed by the covalent bond of a pentose, a nitrogen base and three phosphate groups.

The term nucleoside triphosphates includes deoxynucleoside triphosphates (dNTPs) such as, for example, but not limited to dATP, dCTP, dITP, dUTP, dGTP, dTTP, or derivatives thereof. Preferably, the deoxynucleoside triphosphates are dATP, dTTP, dGTP and dCTP. Still more preferably, these four dNTPs are in equimolar conditions. In a preferred embodiment of this aspect of the invention, the deoxynucleoside triphosphates are at a concentration between 100 μM and 800 μM. In a more preferred embodiment, the deoxynucleoside triphosphates are at a concentration between 200 μM and 600 μM. In a still more preferred embodiment, the deoxynucleoside triphosphates are at a concentration of approximately 500 μM.

The term nucleoside triphosphates also includes dideoxynucleoside triphosphates (ddNTPs) such as, for example, but not limited to, ddATP, ddCTP, ddITP, ddUTP, ddGTP, ddTTP, or derivatives thereof.

In some preferred embodiments of the method for replicating, amplifying or sequencing of the invention, at least one nucleoside triphosphate or one primer is labelled by means of techniques well known in the state of the art. The labelled nucleotide can be, for example, a deoxynucleoside triphosphate or a dideoxynucleoside triphosphate. Detectable labels include, for example, radioactive isotopes, fluorescent labels, chemiluminescent labels, bioluminescent labels or enzymatic labels.

As used in the present description, the term “template DNA” relates to a DNA molecule that can serve as a substrate for synthesizing a complementary DNA strand; i.e., it relates to a DNA molecule to be replicated, amplified or sequenced. In a preferred embodiment the template DNA is plasmid DNA. In another preferred embodiment, the template DNA is genomic DNA.

Replicating, amplifying or sequencing of the template DNA is carried out in primer extension conditions. The expression “primer extension conditions” refers to the conditions in which the template DNA-dependent synthesis initiated in a primer can take place.

The template DNA synthesis according to the method for replicating, amplifying or sequencing of the present invention can take place by means of a thermal cycling process or at an essentially constant temperature.

“Isothermal conditions” is understood as essentially constant temperature. Preferably, the template DNA synthesis according to the method for replicating, amplifying or sequencing of the present invention takes place at an essentially constant temperature. More preferably, at an essentially constant temperature between 25 and 40° C., and still more preferably at approximately 30° C.

A large number of methods allowing DNA amplification are known in the state of the art. Some methods require a thermal cycling process such as, for example, but not limited to the polymerase chain reaction (PCR). Other methods do not require a thermal cycling process, rather they are performed at a essentially constant temperature such as, for example, but not limited to the rolling circle amplification (RCA), the multiple detachment amplification (MDA), the strand displacement amplification (SDA) or the loop mediated amplification (LAMP). The amplification of a template DNA according to the method of the present invention can take place by means of a thermal cycling process or at an essentially constant temperature.

Preferably, the amplification of the template DNA according to the method for amplifying of the present invention takes place by means of rolling circle amplification (RCA), by means of multiple detachment amplification (MDA), strand displacement amplification (SDA) or loop mediated amplification (LAMPA).

Another aspect of the present invention relates to a kit or device comprising elements suitable for carrying out the method for replicating, amplifying or sequencing of the present invention.

Another aspect of the present invention relates to a kit for carrying out the method for replicating, amplifying or sequencing of the present invention comprising:
a) a φ29 type DNA polymerase,
b) polyoxyethylenated sorbitan monolaurate,
c) an ammonium salt,
d) a buffer, and
e) magnesium chloride.

Preferably, said ammonium salt is selected from the list comprising: ammonium sulfate, ammonium chloride or ammonium acetate.

In a preferred embodiment of this aspect of the invention, the kit further comprises a potassium salt. Preferably, said potassium salt is potassium chloride or potassium acetate.

In a preferred embodiment of this aspect of the invention, the kit further comprises a primer. In a more preferred embodiment, the primer is an arbitrary primer which is protected against the action of 3′-5′ exonucleases.

In a preferred embodiment of this aspect of the invention, the kit further comprises nucleoside triphosphates. For example, in a more preferred embodiment of this aspect of the invention, the kit further comprises deoxynucleoside triphosphates and/or a dideoxynucleoside triphosphate.

In a preferred embodiment of this aspect of the invention, the kit comprises at least one nucleoside triphosphate or one labelled primer. The labelled nucleoside can be, for example, a deoxynucleoside triphosphate or a dideoxynucleoside triphosphate.

The kit can further include, without any form of limitation, buffers, agents to prevent contamination, etc. On the other hand, the kit can include all the supports and recipients necessary for putting it into practice and for its optimization. Preferably, the kit further comprises the instructions for carrying out the method of the invention.

Throughout the description and claims, the word “comprises” and its variants do not exclude other technical features, additives, components or steps. For the persons skilled in the art, other objects, advantages and features of the invention will be inferred in part from the description and in part from the practice of the invention. The following drawings and examples are provided by way of illustration and do not limit the present invention.

### EXAMPLES

### Optimization of the Experimental Conditions for Amplifying Multiple-Primer DNA by the φ29 DNA Polymerase

The following specific examples provided in this patent document serve to illustrate the nature of the present invention. These examples are only included for illustrative purposes and must not be interpreted as limitations to the invention claimed herein. Therefore, the examples described below illustrate the invention without limiting the field of application thereof.

It has been shown that the φ29 DNA polymerase amplifies 10⁴-10⁶ times starting from several picograms of circular DNA. For this purpose a reaction buffer containing 40 mM tris-HCl, pH 7.5, 50 mM KCl and 10 mM MgCl₂ (hereinafter Buffer A) was used. After testing the influence of different detergent and salt conditions on the DNA amplification capacity of the φ29 DNA polymerase, it is found that the simultaneous addition of φ0.025% Tween® 20 and 45 mM (NH)₂SO₄ to the Buffer A highly improve the amplification of the limited amounts of provided DNA.

Reaction conditions for amplifying plasmid DNA.—The incubation mixture contained 12.5 μl of buffer A, 50 μM of hexamers protected against the action of the 3′-5′ exonuclease, 500 μM of each of the deoxynucleoside triphosphates (dCTP, dGTP, dTTP and dATP), the indicated amounts of a plasmid DNA (with a size of 4.2 kbp) and, where indicated, 45 mM (NH₄)₂SO₄ or φ0.025% Tween® 20 or a combination of both was added. The DNA was denatured by incubation at 95° C. for 3 minutes and subsequent cooling in ice for 5 min. The reaction was initiated upon adding 50 nM φ29 DNA polymerase and it was stopped after the incubation at 30° C. by means of heating to 65° C. for 10 min. To analyze the results, 1 μl samples were taken from the reactions, the amplified DNA was digested with the EcoRI restriction endonuclease and was subjected to electrophoresis in φ0.7% agarose gel. The DNA was detected by means of staining the gels with ethidium bromide.

Reaction conditions for amplifying genomic DNA.—The incubation mixture contained 12.5 μl of buffer A, 45 mM (NH₄)₂SO₄, φ0.025% Tween® 20, 50 μM of hexamers protected against the action of the 3′-5′ exonuclease, 500 μM of each of the deoxynucleoside triphosphates (dCTP, dGTP, dTTP and dATP) and the indicated amounts of Bacillus subtilis genomic DNA (with a size of 4 Mpb). The DNA was denatured by incubation at 95° C. for 3 minutes and subsequent cooling in ice for 5 min. The reaction was initiated upon adding 50 nM φ29 DNA polymerase and it was stopped after the incubation at 30° C. by means of heating to 65° C. for 10 min. To analyze the results, 1 μl samples were taken from the reactions and were subjected to electrophoresis in φ0.7% agarose gel. The DNA was detected by means of staining the gels with ethidium bromide.

FIG. 1 shows the effect of adding 45 mM (NH₄)₂SO₄ and φ0.025% Tween® 20 in the amplification of small amounts of provided plasmid DNA. As shown, the φ29 DNA polymerase did not give any amplification product detectable with the standard Buffer A when 100 fg of provided DNA were used. In these reaction conditions, the addition of φ0.025% Tween® 20 in the absence of DNA caused the appearance of trace DNA products, most probably as a consequence of the nonspecific DNA amplification caused by the hybridization and elongation of the random hexamer primers. The same trace was observed with 10 fg of provided DNA. However, in the presence of 100 fg of provided DNA, the addition of φ0.025% Tween® 20 allowed the φ29 DNA polymerase to produce a detectable amount of amplified plasmid. The total production of specific or nonspecific amplified DNA indicates that the addition of φ0.025% Tween® 20 to the Buffer A powers the amplification capacity of the φ29 DNA polymerase. An effect similar with the NP40 detergent was observed. Contrarily, other analyzed detergents such as Triton X100 and Triton X114 did not power the amplification capacity of the φ29 DNA polymerase (not shown). The simultaneous addition of φ0.025% Tween® 20 and 45 mM (NH₄)₂SO₄to the buffer A has two consequences in the yield and the specificity of the amplified products: 1) DNA amplification in the absence of provided DNA was not detected; 2) several μg of plasmid DNA of unit length were obtained by amplification even when the provided amount of DNA was as low as 10 fg. As control, the addition of 45 mM (NH₄)₂SO₄to the Buffer A did not produce any improvement in the amplification capacity of the φ29 DNA polymerase.

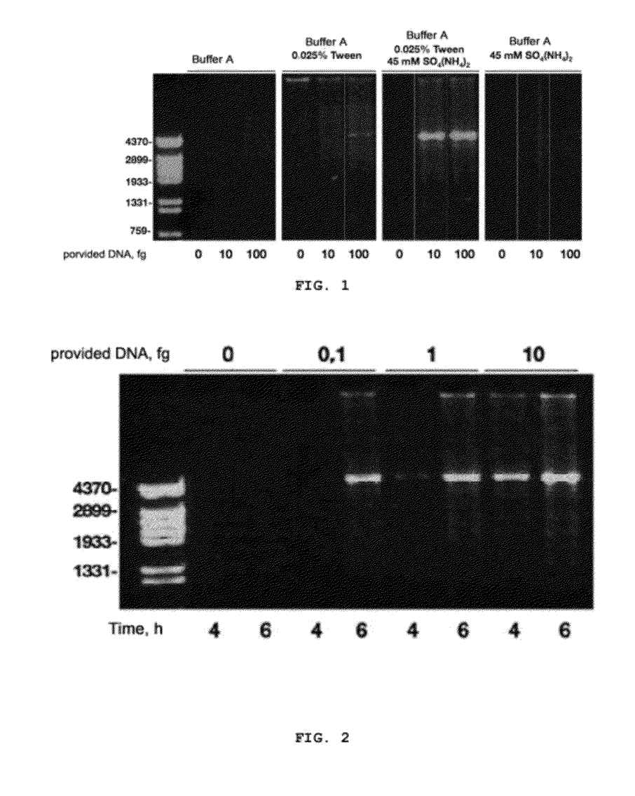

Therefore, it can be concluded that the simultaneous addition of φ0.025% Tween® 20 and 45 mM (NH₄)₂SO₄ to the Buffer A (hereinafter Buffer B) produces a clear optimization of the experimental conditions for carrying out the amplification with multiple priming of circular DNA by the φ29 DNA polymerase, both being absolutely necessary reactants to amplify limited amounts (10 fg) of provided DNA. In fact, as can be seen in FIG. 2, the use of Buffer B allowed the φ29 DNA polymerase to synthesize micrograms of DNA by using a provided amount of plasmid as low as φ0.1 fg (˜24 molecules) after 6 hours of reaction. As quality control, the digestion of the amplification products with EcoRI generated linear dsDNA fragments of 4.2 kb which indicated that the amplification products were really tandem repeats of the original plasmid. Again, the Buffer B also prevented the nonspecific DNA amplification (see in FIG. 2 the lanes corresponding to the reactions carried out without provided DNA).

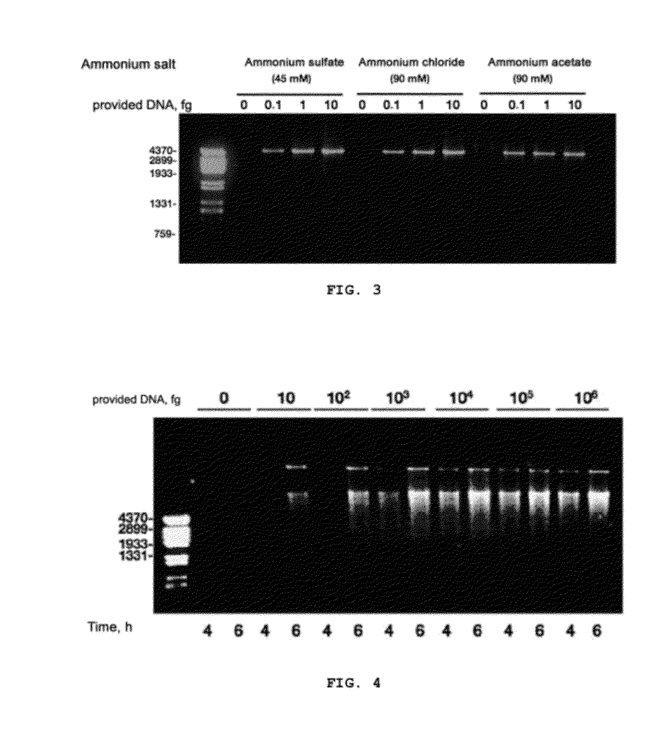

FIG. 3 shows the effect of the ammonium ions and φ0.025% Tween® 20 in improving the amplification of small amounts of plasmid DNA. The assay was carried out in the previously mentioned conditions in the presence of φ0.025% Tween® 20 and the indicated ammonium salt. As can be observed in FIG. 3 both the NH₄Cl and the NH₄CH₃COO had a similar effect to the (NH₄)₂SO₄ both in the yield and in the specificity of the amplified products. This result indicates that the aforementioned effect of the (NH₄)₂SO₄ in the amplification of limiting amounts of plasmid DNA is due to the NH₄ ions.

To determine if the optimized conditions described above also applied to the amplification of genomic DNA, the same type of assays performed in the presence of limited concentrations of B. subtilis genomic DNA (4 Mpb in length) was carried out. As shown in FIG. 4, the presence of φ0.025% Tween® 20 and 45 mM (NH₄)₂SO₄ in the buffer B, on the one hand prevented the nonspecific DNA amplification (lanes without provided DNA), and on the other hand, allowed the φ29 DNA polymerase to give detectable and specific genomic DNA amplification even when 10 fg of provided DNA were used, i.e., an amount 10⁶ times lower than that recommended in the current commercial genomic amplification kits.

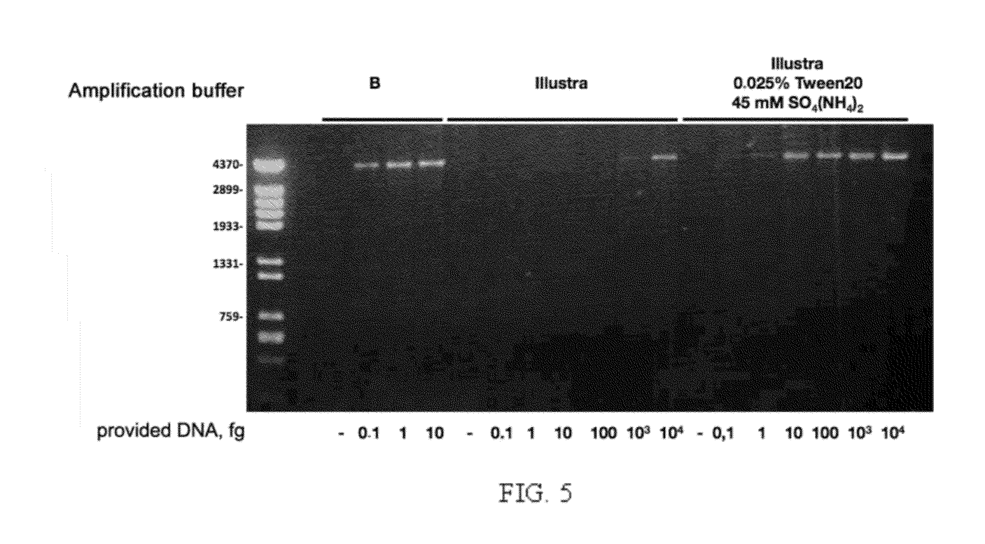

To determine if the simultaneous addition of φ0.025% Tween® 20 and 45 mM (NH₄)₂SO₄ increases the amplification efficiency of the current commercial kits for amplifying DNA based on the φ29 DNA polymerase, the same type of plasmid DNA amplification assays described in FIGS. 1, 2 and 3 was carried out. FIG. 5 shows the significant improvements depicted by the addition of φ0.025% Tween® 20 and 45 mM (NH₄)₂SO₄ to the current reaction buffer of the Illustra kit (GE HealthCare). As can be observed, by following the recommendations of the supplier, with the Illustra kit only amounts of plasmid provided equal to or greater than 10 pg can be amplified in a detectable manner in agarose gel. Contrarily, the simultaneous addition of φ0.025% Tween® 20 and 45 mM (NH₄)₂SO₄ to the reaction buffer of the Illustra kit significantly reduces the needed amount of DNA which can be amplified, amplification products from provided 1 fg of plasmid DNA being observed, involving an improvement of four orders of magnitude in the amplification.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method for replicating, amplifying or sequencing a template DNA which comprises contacting said DNA with a reaction mixture comprising, at least:
a) a φ29 type DNA polymerase,
b) polyoxyethylenated sorbitan monolaurate,
c) an ammonium salt,
d) a buffer,
e) magnesium chloride,
f) a primer, and
g) nucleoside triphosphates.

2. The method according to claim 1, wherein the reaction mixture further comprises a potassium salt.

3. The method according to claim 2, wherein the potassium salt is potassium chloride or potassium acetate.

4. The method according to 1, wherein the polyoxyethylenated sorbitan monolaurate is in a proportion between φ0.003% and φ0.1% of the total volume of the reaction.

5. The method according to claim 4, wherein the polyoxyethylenated sorbitan monolaurate is in a proportion between φ0.006% and φ0.05% of the total volume of the reaction.

6. The method according to claim 5, wherein the polyoxyethylenated sorbitan monolaurate is in a proportion between φ0.01% and φ0.03% of the total volume of the reaction.

7. The method according to claim 1, wherein the ammonium salt is selected from the group consisting of: ammonium sulfate, ammonium chloride and ammonium acetate.

8. The method according to claim 7, wherein the ammonium salt is ammonium sulfate.

9. The method according to claim 8, wherein the ammonium sulfate is at a concentration between 30 mM and 60 mM.

10. The method according to claim 9, wherein the ammonium sulfate is at a concentration between 40 mM and 50 mM.

11. The method according to claim 7, wherein the ammonium salt is ammonium chloride or ammonium acetate.

12. The method according to claim 11, wherein the ammonium chloride or the ammonium acetate is at a concentration between 60 mM and 120 mM.

13. The method according to claim 12, wherein the ammonium chloride or the ammonium acetate is at a concentration between 80 mM and 100 mM.

14. The method according to claim 1, wherein the φ29 type DNA polymerase is selected from the DNA polymerases isolated from the following phages: φ29, Cp-1, PRD-1, φ15, φ21, PZE, PZA, Nf, M2Y, B103, GA-1, SFS, Cp-5, Cp-7, PR4, PRS, PR722, L17 and ABV.

15. The method according to claim 14, wherein the φ29 type DNA polymerase has an amino acid sequence having an identity of at least 80% with the SEQ ID NO: 1.

16. The method according to claim 15, wherein the φ29 type DNA polymerase has an amino acid sequence having an identity of at least 90% with the SEQ ID NO: 1.

17. The method according to claim 16, wherein the φ29 type DNA polymerase has the amino acid sequence SEQ ID NO: 1.

18. The method according to 1, wherein the φ29 type DNA polymerase has a modification in the exonuclease domain and wherein said modified DNA polymerase has less than 10% of exonuclease activity than the corresponding naturally occurring DNA polymerase.

19. The method according to claim 18, wherein the modified φ29 type DNA polymerase has less than 1% of exonuclease activity than the corresponding naturally occurring DNA polymerase.

20. The method according to claim 19, wherein the modified φ29 type DNA polymerase lacks detectable exonuclease activity with respect to the corresponding naturally occurring DNA polymerase.

21. The method according to claim 1, wherein the buffer is tris-hydrochloric, tris-acetic or HEPES.

22. The method according to claim 1, wherein the buffer is at a pH between 7 and 8.5.

23. The method according to claim 1, wherein the magnesium chloride is at a concentration between 2 mM and 20 mM.

24. The method according to claim 23, wherein the magnesium chloride is at a concentration between 5 mM and 15 mM.

25. The method according to claim 2, wherein the potassium chloride or the potassium acetate is at a concentration between 30 mM and 70 mM.

26. The method according to claim 25, wherein the potassium chloride or the potassium acetate is at a concentration between 40 mM and 60 mM.

27. The method according to claim 1, wherein the nucleoside triphosphates are dCTP, dGTP, dTTP and dATP.

28. The method according to claim 27, wherein the dCTP, dGTP, dTTP and dATP nucleoside triphosphates are in equimolar amounts.

29. The method according to claim 1, wherein the primer is arbitrary and is protected against the action of exonucleases.

30. The method according to claim 1, wherein the template DNA is plasmid DNA.

31. The method according to claim 1, wherein the template DNA is genomic DNA.

32. The method according to claim 1, wherein the amplification is performed at an essentially constant temperature between 25 and 40° C.

33. The method for amplifying a template DNA according to claim 1, wherein the amplification takes place by means of rolling circle amplification (RCA), multiple displacement amplification (MDA), strand displacement amplification (SDA) or loop mediated amplification (LAMPA).

34. The method according to claim 1, wherein at least one nucleoside triphosphate or one primer is labelled.

35. A kit for carrying out a method for replicating, amplifying or sequencing a template DNA comprising:
a) a φ29 type DNA polymerase,
b) polyoxyethylenated sorbitan monolaurate,
c) an ammonium salt,
d) a buffer, and
e) magnesium chloride.

36. A kit according to claim 35, further comprising a potassium salt.

37. A kit according to claim 35, further comprising a primer.

38. A kit according to claim 37, wherein the primer is arbitrary and is protected against the action of exonucleases.

39. A kit according to claim 35, further comprising nucleoside triphosphates.

40. A kit according to claim 37 wherein at least one nucleoside triphosphate or one primer is labelled.
